(19) United States
(12) Patent Application Publication
(10) Pub. No.: US 2025/0230112 A1
(43) Pub. Date: Jul. 17, 2025

(54) PROCESSES FOR CONVERTING SATURATED POLYETHYLENE TO ALKENE PRODUCTS

(71) Applicants: Dow Global Technologies LLC, Midland, MI (US); The Regents of the University of California, Oakland, CA (US); The Board of Trustees of the University of Illinois, Urbana, IL (US)

(72) Inventors: Ivan A. Konstantinov, Manvel, TX (US); Rafael Huacuja, Rosharon, TX (US); Alex J. Nett, Midland, MI (US); Mari S. Rosen, Houston, TX (US); Sean W. Ewart, Pearland, TX (US); Garrett Strong, Goleta, CA (US); Lijun Gao, Santa Barbra, CA (US); Nicholas Wang, Pittsburg, PA (US); Vanessa DaSilva, Urbana, IL (US); Damien S. Guironnet, Urbana, IL (US); Susanna Scott, Goleta, CA (US)

(73) Assignees: Dow Global Technologies LLC, Midland, MI (US); The Regents of the University of California, Oakland, CA (US); The Board of Trustees of the University of Illinois, Urbana, IL (US)

(21) Appl. No.: 18/875,480

(22) PCT Filed: Jun. 14, 2023
(86) PCT No.: PCT/US2023/068415

§ 371 Date: Dec. 16, 2024

Related U.S. Application Data
(60) Provisional application No. 63/353,320, filed on Jun. 17, 2022.

(51) Int. Cl. C07C 6/04; B01J 6/00; B01J 23/42; B01J 31/12; B01J 35/61; B01J 35/63; B01J 37/02; B01J 37/08; C07C 5/373

(52) U.S. Cl. CPC C07C 6/04; B01J 6/001; B01J 23/42; B01J 31/12; B01J 35/615; B01J 35/635; B01J 35/638; B01J 37/0201; B01J 37/08; C07C 5/373; B01J 2231/543; B01J 2231/766; C07C 2523/42; C07C 2531/12

(57) ABSTRACT

This disclosure relates to processes for converting saturated polyethylene to at least an alkene product. The processes comprise contacting the saturated polyethylene with three or more catalyst components in a reactor, the reactor comprising an alkene reactant. The three or more catalyst components comprise a metathesis catalyst component, an isomerization catalyst component, and a dehydrogenation catalyst component. Contacting causes at least a portion of the saturated polyethylene to undergo dehydrogenation reactions to form unsaturated polyethylene and at least a portion of the unsaturated polyethylene, or products derived therefrom, to undergo metathesis reactions and isomerization reactions to produce an effluent comprising at least the alkene product.

### CROSS-REFERENCE TO RELATED APPLICATIONS

This application claims the benefit of U.S. Provisional Application Ser. No. 63/353,320 filed Jun. 17, 2022, the entire disclosure of which is hereby incorporated herein by reference.

### BACKGROUND

### Field

The present disclosure relates to chemical processing of hydrocarbons. In particular, the present disclosure relates to processes for converting ethylene-containing materials, such as polyethylene into smaller desirable hydrocarbon products.

### Technical Background

For a number of industrial applications, hydrocarbons are used, or are starting materials used, to produce plastics, fuels, and various downstream chemicals. Such hydrocarbons include alkenes, such as ethene, propene and butenes (also commonly referred to as ethylene, propylene, and butylenes, respectively). A variety of processes for producing these lower hydrocarbons have been developed, including petroleum cracking and various synthetic processes. Polyethylene (PE), the most widely used plastic in the world, can be made into a wide variety of products. However, processes for the recycling of polyethylene into smaller monomers, such as propylene, is desired. Conventional efforts for chemical recycling of polyethylene have generally used pyrolysis and high-temperature thermal degradation. These processes are highly energy intensive and are plagued by low selectivity of desired products and generation of greenhouse gases (e.g. CO₂, CH₄).

### SUMMARY

Embodiments of the present disclosure address these and other needs by providing processes for converting polyethylene into alkene products. The processes described herein may enable three or more catalyst components in a reactor system to conduct a plurality of different chemical reactions, such as combinations of dehydrogenation, metathesis, and isomerization for producing alkene products from saturated polyethylene and an alkene reactant, for example.

According to one or more other aspects of the present disclosure, a process for converting saturated polyethylene to at least an alkene product of chemical formula CₘH₂ₘ, the process comprising contacting the saturated polyethylene with three or more catalyst components in a reactor, the reactor comprising an alkene reactant of chemical formula CₙH₂ₙ, where m is an integer from 3 to 20 and n is an integer from 2 to 20. The three or more catalyst components comprise a metathesis catalyst component, an isomerization catalyst component, and a dehydrogenation catalyst component. Contacting causes at least a portion of the saturated polyethylene to undergo dehydrogenation reactions to form unsaturated polyethylene and at least a portion of the unsaturated polyethylene, or products derived therefrom, to undergo metathesis reactions and isomerization reactions to produce an effluent comprising at least the alkene product of chemical formula CₘH₂ₘ.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### BRIEF DESCRIPTION OF THE DRAWING

FIG. 1 schematically depicts a reactor, in accordance with one or more embodiments of the present disclosure.

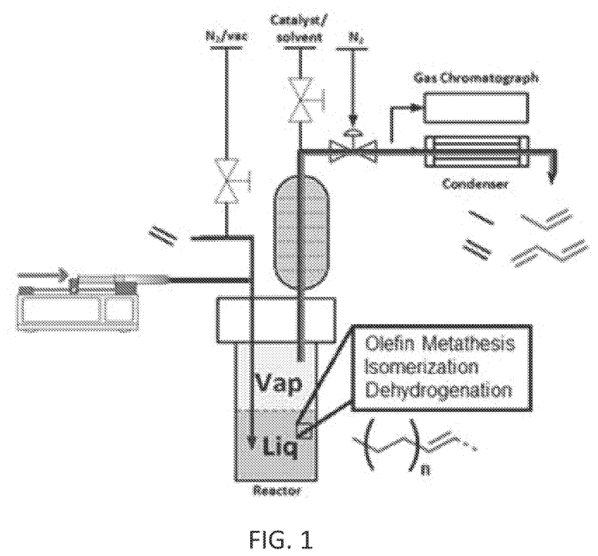

For the purpose of describing the simplified schematic illustration and description of FIG. 1, the numerous valves, temperature sensors, electronic controllers, and the like that may be employed and well known to those of ordinary skill in the art of certain chemical processing operations are not included. Further, accompanying components that are often included in typical chemical processing operations, carrier gas supply systems, pumps, compressors, furnaces, or other subsystems are not depicted. It should be understood that these components are within the spirit and scope of the present embodiments disclosed. However, operational components, such as those described in the present disclosure, may be added to the embodiments described in this disclosure

### DETAILED DESCRIPTION

Some conventional processes for converting polyethylene to smaller products may use separate catalysts isolated in separate catalyst zones, such as by charging each of the separate catalysts to a separate reactor, which can increase the initial capital cost of the reaction system. In contrast, processes disclosed herein can enable tandem catalysis of polyethylene by contacting the polyethylene with mutually compatible catalyst components to produce the desired alkene products. The catalytic depolymerization of polyethylene under mild reaction conditions provides an advantageous and sustainable alternative for the production of hydrocarbon feedstock, monomers or other useful chemicals.

Reference will now be made in detail to embodiments of processes for converting saturated polyethylene to alkene products in a reactor. As used herein, “saturated polyethylene” refers to a compound comprising the chemical formula CₓH₂ₓ₊₂, where x is an integer of at least 10, and where the carbon-carbon bonds are single bonds. In embodiments, the saturated polyethylene can include branched polyethylene. In embodiments, the saturated polyethylene can include linear low-density polyethylene (LLDPE), low density polyethylene (LDPE), or combinations thereof. In embodiments, the saturated polyethylene can include a chemical compound comprising the chemical formula CH₃(C₂H₄)ₓCH₃. In embodiments, the saturated polyethylene comprises CₓH₂ₓ₊₂, where x is an integer of greater than or equal to 10, greater than or equal to 12, or even greater than or equal to 15. In embodiments, the saturated polyethylene can have a number average molecule weight (Mₙ) of from 150 g/mol to 1,000,000 g/mol. In embodiments, the saturated polyethylene can be a waste stream, or product derived therefrom, of a hydrocarbon processing system.

In embodiments, the reactor comprises an alkene reactant. In embodiments, the alkene reactant has a chemical formula of CₙH₂ₙ, where n is an integer from 2 to 20. For example, the alkene reactant can have a chemical formula of CₙH₂ₙ, where n is an integer from 2 to 15, from 2 to 10, from 2 to 5, from 2 to 4, or from 2 to 3. In embodiments, the alkene reactant can comprise ethylene, propylene, butenes, pentenes, or combinations thereof. In embodiments, the alkene reactant can be selected from the group consisting of ethylene, propylene, butenes, pentenes, and combinations thereof. In embodiments, the alkene reactant can comprise ethylene. In embodiments, the alkene reactant can consist essentially of or consist of ethylene. In embodiments, the alkene reactant can comprise ethylene and butenes. In embodiments, the alkene reactant can consist essentially of or consist of ethylene and butenes.

In embodiments, the saturated polyethylene can be contacted with three or more catalyst components in a reactor. As used herein, “catalyst components” refers to any substance which increases the rate of a specific chemical reaction. Catalyst components and the catalyst compositions made with the catalyst components described in this disclosure may be utilized to promote various reactions, such as, but not limited to, dehydrogenation, metathesis, isomerization, or combinations of these. In embodiments, a catalyst composition can include at least one catalyst component, at least two catalyst components or at least three catalyst components. As used herein, “catalyst composition” refers to a solid particulate comprising at least one catalyst component. The catalyst composition can further comprise a catalyst support material.

In embodiments the catalyst components can include a dehydrogenation catalyst component, a metathesis catalyst component, and an isomerization catalyst component. Without intending to be bound by any particular theory, it is believed that the dehydrogenation catalyst can introduce an unsaturation in the carbon chain of the saturated polyethylene to produce unsaturated polyethylene. It is believed that the metathesis catalyst, in the presence of the alkene reactant, can break the carbon chain of the unsaturated polyethylene to produce two products that each have a terminal unsaturation, and further metathesis of the terminally unsaturated polyethylene intermediate product with the alkene reactant may be unproductive to further break the carbon chain. It is believed that the isomerization catalyst component can convert the terminal unsaturation to an internal unsaturation, and the isomerized product can be further broken into two products in the presence of the metathesis catalyst component and the alkene reactant. This cycle can continue until the desired product or group of products is produced from the process.

In embodiments, the dehydrogenation catalyst component can be operable to convert saturated polyethylene to unsaturated polyethylene. In embodiments, the dehydrogenation catalyst component can cause saturated polyethylene, or products derived therefrom, to have additional unsaturations along the polyethylene backbone. In embodiments, the dehydrogenation catalyst component can cause the saturated polyethylene or products derived therefrom to undergo transfer dehydrogenation. In embodiments, the dehydrogenation catalyst component can include one or more elements selected from International Union of Pure and Applied Chemistry (IUPAC) groups 5-10. In embodiments, the dehydrogenation catalyst component can comprise platinum, iridium, ruthenium, rhenium, or combinations thereof. In embodiments, the dehydrogenation catalyst component is selected from the group consisting of platinum, iridium, ruthenium, rhenium, and combinations thereof.

In embodiments, the metathesis catalyst component in combination with the alkene reactant, such as ethylene, can be operable to break the unsaturated polyethylene chain into two species. In embodiments, the metathesis catalyst component can break alkene products derived from the saturated polyethylene. In embodiments, the metathesis catalyst component can include one or more elements selected from International Union of Pure and Applied Chemistry (IUPAC) groups 5-10. In embodiments, the metathesis catalyst component can comprise rhenium, ruthenium, tungsten, molybdenum, vanadium, or combinations thereof. In embodiments, the metathesis catalyst component can be selected from the group consisting of rhenium, ruthenium, tungsten, molybdenum, vanadium, and combinations thereof. In embodiments, the metathesis catalyst component can comprise methyltrioxorhenium (MTO).

In embodiments, the isomerization catalyst component can be operable to move an unsaturation on unsaturated polyethylene, or an unsaturation on products derived therefrom, from one position on the backbone to a different position. For instance, in embodiments, the isomerization catalyst component can move an unsaturation in a terminal position of the unsaturated polyethylene to an internal position. In embodiments, the isomerization catalyst component can include one or more elements selected from International Union of Pure and Applied Chemistry (IUPAC) groups 5-10. In embodiments, the isomerization catalyst component can comprise alumina, silica, iridium, palladium, ruthenium or combinations thereof. In embodiments, the isomerization catalyst component can be selected from the group consisting of alumina, silica, iridium, palladium, ruthenium, and combinations thereof. In embodiments, the isomerization catalyst component can include modified alumina, modified silica, or combinations thereof. For instance, in embodiments, the isomerization catalyst component can include, but not be limited to, chlorinated alumina, gamma-alumina, chlorinated silica, or combinations thereof. In embodiments, the isomerization catalyst component can comprise [tert-butyl-POCOP]Ir[C₂H₄].

In embodiments, the reactor may comprise one or more catalyst compositions that comprise the three or more catalyst components. For instance, in embodiments, a catalyst composition can comprise a metathesis catalyst component and an isomerization catalyst component. In embodiments, a catalyst composition can comprise a dehydrogenation catalyst component and an isomerization catalyst component. In embodiments, a catalyst composition can comprise a metathesis catalyst component and an isomerization catalyst component. In embodiments, a catalyst composition can comprise a dehydrogenation catalyst component and a metathesis catalyst component. In embodiments, a catalyst composition can comprise a dehydrogenation catalyst component, a metathesis catalyst component, and an isomerization catalyst component. In embodiments, a catalyst composition can comprise a dehydrogenation catalyst component, a metathesis catalyst component, or an isomerization catalyst component. In embodiments, the reactor can comprise a first catalyst composition comprising a metathesis catalyst component and an isomerization catalyst component. For instance, in embodiments, the reactor can comprise a first catalyst component, where the first catalyst component is MTO on alumina. In embodiments, the reactor can comprise a second catalyst composition comprising a dehydrogenation catalyst component and an isomerization catalyst component. For instance, in embodiments the reactor can comprise a second catalyst component, where the second catalyst component can comprise platinum on alumina or platinum on silica. In embodiments, a first catalyst composition comprising MTO on alumina and a second catalyst composition comprising platinum on alumina can contact the saturated polyethylene in the reactor. In other embodiments, a first catalyst composition comprising MTO on alumina and a second catalyst composition comprising [tert-butyl-POCOP]Ir[C₂H₄]can contact the saturated polyethylene in the reactor.

In embodiments, the catalyst composition is designated by a weight percentage of the one or more elements selected from International Union of Pure and Applied Chemistry (IUPAC) groups 5-10. In embodiments, the first catalyst composition can comprise less than or equal to 15 wt. % of any one of the elements selected from the IUPAC groups 5-10 based on the total weight of the first catalyst composition. For instance, in embodiments, the first catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the first catalyst composition. In embodiments, the first catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the first catalyst composition. In embodiments, the first catalyst composition can comprise any one of the elements selected from the IUPAC groups 5-10 in an amount of from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the first catalyst composition.

In embodiments, the second catalyst composition can comprise less than or equal to 15 wt. % of any one of the elements selected from the IUPAC groups 5-10 based on the total weight of the second catalyst composition. For instance, in embodiments, the second catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the second catalyst composition. In embodiments, the second catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the second catalyst composition. In embodiments, the second catalyst composition can comprise any one of the elements selected from the IUPAC groups 5-10 in an amount from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the second catalyst composition.

It should be understood that according to embodiments, the catalyst composition may be made by methods that lead to the desired composition. Some non-limiting instances include incipient wetness impregnation, or vapor phase deposition of metal precursors (either organic or inorganic in nature), followed by their controlled decomposition.

In embodiments, contacting the saturated polyethylene with three or more catalyst components in a reactor comprising an alkene reactant can cause at least a portion of the saturated polyethylene to undergo dehydrogenation reactions to form unsaturated polyethylene and at least a portion of the unsaturated polyethylene, or products derived therefrom, to undergo metathesis reactions and isomerization reactions to produce an effluent comprising at least the alkene product of chemical formula CₘH₂ₘ. For instance, the contacting of the saturated polyethylene with the dehydrogenation catalyst component can dehydrogenate the saturated polyethylene, which introduces at least one unsaturation into the backbone of the polyethylene to form unsaturated polyethylene. In embodiments, the unsaturated polyethylene can contact the metathesis catalyst component in the presence of the alkene reactant to break the unsaturated polyethylene to form two products, where each product comprises a terminal unsaturated polyethylene. The terminal unsaturated polyethylene can contact the isomerization catalyst component to cause the unsaturation to move in the terminal unsaturated polyethylene from a terminal position to an internal position to form an internal unsaturated polyethylene. Without intending to be bound by any particular theory, it is believed that the internal unsaturated polyethylene can undergo further metathesis reactions by contacting the metathesis catalyst component in the presence of the alkene reactant. It is believed that the products derived from the unsaturated polyethylene that contact both the metathesis catalyst component and the isomerization catalyst component in the presence of the alkene reactant can continue to cycle between metathesis and isomerization reactions to produce smaller alkene products, such as compounds of chemical formula CₘH₂ₘ, where m is an integer from 3 to 20, for instance, propylene. In embodiments, the reaction time can be increased to produce an effluent comprising smaller alkene products, as increased reaction time will allow additional metathesis and isomerization reaction cycles.

In embodiments, the reactor can be any reactor useful for causing the polyethylene to contact the three or more catalyst components in the presence of the alkene reactant and cause the catalytic reactions to proceed, such as a batch reactor, a fixed-bed reactor, a fluidized bed reactor, a continuous stirred tank reactor, a tubular plug flow reactor, a reactive extruder, or combinations thereof. In embodiments two or more reactors can be used, such as two or more reactors in series. In embodiments, the reactor can comprise a reaction zone where the contacting and the catalytic reactions can occur. In embodiments, the three or more catalyst components can be in the same reaction zone. In other embodiments, the reactor can comprise two or more reaction zones. In embodiments, the reactor can include additional processing of the reactants, such as processing of the alkene reactant, the saturated polyethylene, and/or the catalyst components. In embodiments, the effluent comprising one or more products from the catalytic reactions can be further processed, such as separation of one or more products from the effluent. For instance, in embodiments, propylene can be separated from the effluent.

In embodiments, a pressure of the alkene reactant in the reactor, such as in the reaction zone during the contacting can be from 0 pounds per square inch gauge (psig) to 3000 psig. For instance, a pressure of the alkene reactant can be of from 0 psig to 3000 psig, from 0 psig to 2000 psig, from 0 psig to 1000 psig, from 0 psig to 900 psig, from 0 psig to 800 psig, from 0 psig to 700 psig, from 0 psig to 600 psig, from 0 psig to 500 psig, or from 100 psig to 3000 psig. In some embodiments, the amount of the alkene reactant used can be quantified by the pressure of the alkene reactant in the reactor. In other embodiments, the amount of the alkene reactant can be quantified by a space velocity of the alkene reactant.

In embodiments, a temperature of the reactor, such as in the reaction zone, during the contacting can be less than or equal to 400° C. For instance, a temperature of the reactor during the contacting can be less than or equal to 350° C., less than or equal to 300° C., less than or equal to 250° C., or even less than or equal to 200° C. In embodiments, a temperature of the reactor during the contacting can be of from 50° C. to 400° C., from 50° C. to 350° C., from 50° C. to 300° C., from 50° C. to 250° C., from 50° C. to 200° C., from 60° C. to 400° C., from 60° C. to 350° C., from 60° C. to 300° C., from 60° C. to 250° C., or from 60° C. to 200° C. Without intending to be bound by any particular theory, it is believed that a reduced reactor temperature, such as less than or equal to 400° C., less than or equal to 350° C., less than or equal to 300° C., less than or equal to 250° C., or less than or equal to 200° C., can reduce the formation of undesired side products during the contacting. Further, the reduced operational temperature of the reactor can reduce the energy required for the process, which can also reduce the economic cost of operating.

In embodiments, the contacting causes at least a portion of the saturated polyethylene to undergo catalytic reactions to produce an effluent. In embodiments, the effluent can comprise hydrocarbons having an average molecular weight of from 40 g/mol to 1000 g/mol. In embodiments, the effluent can comprise at least the alkene product of chemical formula CₘH₂ₘ. In embodiments, the alkene product is a compound of chemical formula CₘH₂ₘ, where m is an integer from 3 to 20. For instance, the alkene product can be a compound of chemical formula CₘH₂ₘ, where m is an integer from 3 to 15, from 3 to 10, from 3 to 8, from 3 to 7, from 3 to 6, from 3 to 5, from 3 to 4, or of 3. In embodiments, the alkene product can comprise propylene, butenes, pentenes, or combinations thereof. In embodiments, the alkene product can be selected from the group consisting of propylene, butenes, pentenes, and combinations thereof. In embodiments, the alkene product can consist essentially of, or consist of, propylene, butenes, pentenes, or combinations thereof. In embodiments, the alkene product can consist essentially of, or consist of propylene.

In embodiments, the effluent can comprise at least 1 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, or even at least 60 wt. % of the alkene product.

### EXAMPLES

The various aspects of the present disclosure will be further clarified by the following examples. The examples are illustrative in nature and should not be understood to limit the subject matter of the present disclosure. In Examples 1-7, catalysts according to the present disclosure were prepared. The materials used in the Examples are provided below in Table 1.

TABLE 1

| | | Chemical Description, | |
|---|---|---|---|
| Ingredient | Product | Chemical formula, or | |
| Type | Name | Structure | Source |
| Catalyst | Methyl- | CH₃ReO₃ (MTO), solid | Sigma- |
| precursor | trioxorhenium | crystal, Re concentration | Aldrich |
| | | 71.0-76.0% | |
| Catalyst | Ammonium | H₄NO₄Re, solid | Sigma- |
| precursor | perrhenate | crystal, ≥99% purity | Aldrich |
| Catalyst | gamma- | γ-Al₂O₃, surface area 186 | Strem |
| support | Alumina | m²g⁻¹ pore volume 0.50 | Chemicals, |
| | | cm³g⁻¹, 97% purity | Inc. |
| Catalyst | Sylopol 952 | SiO₂, surface area 249 m²/g,, | Grace |
| support | Silica | pore volume 1.61 mL/g | Division |
| Chlorine | Carbon | CCl₄, anhydrous, ≥99.5% | Sigma- |
| source | tetrachloride | purity, solvent | Aldrich |
| Chlorine | Hydrochloric | HCl, 37% in aqueous solution | EMD |
| source | acid | | Millipore |
| Reactant | Saturated | M_{w} = 37,000 g/mol | Dow |
| | polyethylene | Mₙ = 1,900 | Chemical |
| Reactant | n-octadecane | C₁₈H₃₈, 99%, crystalline | Alfa Aesar |
| | | mass/melt | |
| Metal | trimethyl | C₈H₁₄Pt, 99% | Strem |
| precursor | (cyclo- | | Chemicals, |
| | pentadienyl) | | Inc. |
| | platinum | | |
| Extraction | Carbon | CS₂, ≥99.9% purity, solvent | Fisher |
| solvent | disulfide | | |
| Extraction | Chloroform | CHCl₃, HPLC grade, | EMD |
| solvent | | OmniSolv ®, CX1054-6 | Millipore |
| | | | Corp. |
| Reactant | Ethylene | C₂H₄, Ultra high purity | Airgas |
| | | (UHP), gas UHP, gas, | |
| | | purified with an oxygen/ | |
| | | moisture trap (Supelco) | |
| Oxidizing | Oxygen | O₂, UHP, gas | Airgas |
| gas | | | |
| Carrier | Argon | Ar, UHP, gas | Airgas |
| gas | | | |
| Carrier | Nitrogen | N₂, UHP, gas | Airgas |
| gas | | | |
| Reducing | Hydrogen in | 5% H₂ in Ar, gas | Airgas |
| gas | argon | | |

In examples 8-10, catalytic processes according to the present disclosure were carried out in a batch reactor. Hydrocarbons in the gas fraction product (CI-C₆) were analyzed quantitatively on a Shimadzu GC-2010 gas chromatograph equipped with a capillary column (Supelco Alumina Sulfate plot, 30 m×0.32 mm) and a flame ionization detector (FID). The signal coefficient is dependent on the carbon number for each hydrocarbon species. The injector and detector temperatures were 200° C. The temperature ramp program was as follows: 90° C. (hold 3 min), ramp 10° C./min to 150° C. (hold 20 min). Helium was used as carrier gas. H₂, C₂H₄, and C₂H₆ were quantified on a Shimadzu GC-8AIT gas chromatograph equipped with a packed column (ShinCarbon ST 80/100, 2 m×2 mm) and a thermal conductivity detector (TCD). The linear response of the TCD signal to the injected volumes of H₂, C₂H₄, and C₂H₆ was confirmed using standard gas mixtures. The response factors were obtained as the slopes of fitted lines. The column, injector and detector temperatures were 130° C. The TCD current was 70 mA and the carrier gas pressure was 300 kPa (N₂). Liquid phase products (>C₅) were analyzed on an Agilent 6890N Network Gas Chromatograph equipped with a DB-5 column and an FID detector.

### Example 1. Preparation of CH₃ReO₃/Cl—Al₂O₃ Catalyst Composition

The catalyst composition of Example 1, 4 wt. % CH₃ReO₃/Cl—Al₂O₃, was synthesized using the following procedure: y-Al₂O₃(Strem Chemicals, Inc.) was calcined at 550° C. in air for 4 hours (h), followed by evacuation at 450° C. under dynamic vacuum (10⁻⁴ Torr) overnight. This partially dehydrated and dehydroxylated alumina was chlorinated in a stream of CCl₄-saturated Ar (Airgas, UHP, 10 mL/min) in a fixed bed reactor at 300° C. for 1 h. CCl₄ was distilled prior to use. The resulting Cl—Al₂O₃ was evacuated at 450° C. overnight and modified with CH₃ReO₃ (MTO, Sigma-Aldrich) by vacuum sublimation (ca. 10⁻⁴ Torr) at room temperature to obtain a material containing 4 wt. % MTO and 4 wt. % Cl based on the total weight of the material. Periodically, the solid was shaken vigorously to promote uniform deposition of MTO. After grafting the MTO on the Cl—Al₂O₃, the catalyst was evacuated 30 min at room temperature to remove physisorbed material and the catalyst was stored in a N₂-filled glovebox to prevent deactivation in air.

### Example 2. Preparation of 1.5% Pt/γ-Al₂O₃ Catalyst Composition

The catalyst composition of Example 2, 1.5% Pt/γ-Al₂O₃, was synthesized using the following procedure: γ-Al₂O₃(Strem Chemicals, Inc., 186 m² g⁻¹, pore volume 0.50 cm³ g⁻¹) was calcined in air at 500° C. for 4 h, followed by evacuation (10⁻⁴ Torr) at 450° C. for 12 h. Volatile trimethyl(cyclopentadienyl)platinum (32+1 mg) was deposited onto dry alumina (1.300±0.020 g) by vacuum sublimation (ca. 10⁻⁴ Torr) at room temperature to obtain materials with 1.5 wt % Pt. The reactor was shaken vigorously during the procedure to promote uniform deposition followed by evacuation at room temperature for 1 h to remove physisorbed PtCp(CH₃)₃. The resulting solid was reduced in flowing H₂ (4.0% in Ar, 30 mL/min) as the temperature was ramped to 250° C. at a rate of 2° C./min. The material was held at this temperature for 2 h, then cooled to room temperature and evacuated for 15 min. The reduced catalyst was stored in a N₂-filled glovebox until use to avoid re-oxidation in air.

### Example 3. Preparation of 1.5% Pt/Cl—Al₂O₃ Catalyst Composition

The catalyst composition of Example 3, 1.5% Pt/Cl—Al₂O₃, was synthesized using the following procedure: γ-Al₂O₃(Strem Chemicals, Inc., 186 m² g⁻¹, pore volume 0.50 cm³ g⁻¹) was calcined in air at 500° C. for 4 h. 1.5 g of calcined alumina was impregnated with 0.6 ml aqueous solution containing 45.0 mg of Pt(NH₃)₄(NO₃)₂ and 46 mg HCl (from concentrated HCl), followed by drying in the oven at 80° C. for 2 h and calcination at 500° C. for 3 hours under static air, with a ramp rate of 2° C./min. The resulting material was then reduced under H₂ (5.0% in Ar, 30 mL/min) 280° C. for 2 hours, with a ramp rate of 2° C./min, which was followed by evacuation under ca. 10⁻⁴ Torr at room temperature for 30 minutes. The reduced catalyst was stored in a N₂-filled glovebox until use to avoid re-oxidation in air.

### Example 4. Preparation of 1.5% Pt/SiO₂ Catalyst Composition

The catalyst composition of Example 4, 1.5% Pt/SiO₂, was synthesized using the following procedure: 1.5 g of calcined SiO₂ (Sylopol 952) was impregnated with 1.5 mL of an aqueous solution containing 45.0 mg of Pt(NH₃)₄(NO₃)₂, followed by drying in the oven at 80° C. overnight and calcination at 350° C. for 3 hours under static air, with a ramp rate of 2° C./min. The resulting material was then reduced under 5.0% H₂/Ar at 280° C. for 2 hours, with a ramp rate of 2° C./min. which was followed by evacuation under ca. 10⁻⁴ Torr at room temperature for 30 minutes. The catalyst comprising 1.5 wt. % Pt was stored in a N₂-filled glovebox until use to avoid re-oxidation in air.

### Example 5. Preparation of Re₂O₇/γ-Al₂O₃Catalyst Composition

The catalyst composition of Example 5, Re₂O₇/γ-Al₂O₃, was synthesized using the following procedure: Re₂O₇/γ-Al₂O₃ was prepared by incipient wetness impregnation of γ-Al₂O₃ (Strem Chemicals, Inc.) with ammonium perrhenate to obtain a material containing 10 wt. % Re. Prior to impregnation, γ-Al₂O₃ was calcined at 550° C. for 4 h within 2 h. After impregnation, the dried material was activated by calcination in oxygen at 650° C. at 5° C./min for 8 h. The calcined catalyst was stored in a N₂-filled glovebox until use to avoid deactivation in air.

### Example 6. Preparation of PtRe/SiO₂ Catalyst Composition

The catalyst composition of Example 6, PtRe/SiO₂, was prepared using the following procedure: PtRe/SiO₂ was prepared by incipient wetness impregnation of silica powder with ammonium perrhenate to obtain a material containing 1-5 wt % Re. After impregnation, the material was calcined at 500° C. Pt was deposited on the material by incipient wetness impregnation in toluene with platinum acetylacetonate to obtain a material containing 1-5 wt % Pt. The resulting solid was dried in air at 120° C. for 4 h after which the temperature was increased to 210° C. for 4 h. The material was reduced in H₂ at 150° C. for 1 h. The reduced catalyst was stored in a N₂ atmosphere until use to avoid re-oxidation in air. The PtRe/SiO₂ catalyst was calcined at 500° C. for 4 h followed by reduction with H₂ at 280° C. for 2 h. The heating rate is 2° C./min. After reduction, the catalyst was evacuated 30 min at room temperature to remove physisorbed H₂ and stored in N₂-filled glovebox to prevent deactivation in air.

### Example 7. Preparation of [^{tBu}POCOP]Ir[C₂H₄]Catalyst Composition

The catalyst composition of Example 7, [^{tBu}POCOP]Ir[C₂H₄], was prepared according to “Catalytic Alkane Metathesis by Tandem Alkane Dehydrogenation-Olefin Metathesis” Science 2006, 312, 257-261. [C₆H₃-2,6-[OP(t-Bu)₂]₂]Ir[H][Cl] and NaO-t-Bu were weighed into an oven-dried Schlenk flask in a molar ratio of 1 to 1.2, respectively. The solids were then put under a flow of argon. 40 mL of toluene was added to the flask via syringe, and the resulting suspension was stirred for 10 min at room temperature. Ethylene was bubbled through the solution for 1-2 hours. The solution was cannula-filtered through a pad of Celite, volatiles were evaporated under vacuum, and the resulting red solid was dried under vacuum overnight to afford the product in 60% yield.

### Example 8. Catalytic Conversion of Saturated Polyethylene in a Batch Reactor to Form Alkenes

In Example 8, saturated polyethylene was reacted with ethylene over the catalysts of Example 2 and Example 5 in a 25 mL batch reactor (Parr reactor, Series 4590). In an N₂-filled glovebox, 199 mg of Example 5, 199 mg of Example 2, and 120 mg of saturated polyethylene were loaded into a 25 mL reactor equipped with a pressure gauge and type K thermocouple. Ethylene (99.999%, Airgas) was passed through a moisture/oxygen trap (Supelco) before use. Gas lines were purged of residual air for three 5-min cycles before ethylene was introduced into the reactor. After pressurization, the total pressure was 40 bar. Reactor heating was initiated, and reaction time was tracked after reaching a desired temperature of 200° C. After a reaction time of 24 hours, the reactor was cooled in flowing air. Aliquots of gas from the reactor headspace were taken for GC analysis before venting the rest of the headspace in a fume hood. The remaining solid and liquid was transferred onto a fine glass filter (4.0-5.5 μm) and filtered to remove insoluble material by washing with hot (50° C.) CHCl₃. Soluble hydrocarbons were recovered by evaporating the solvent under reduced pressure (0.1 Torr). The insoluble material, including the catalyst and hydrocarbons insoluble in hot CHCl₃, was recovered from the filter. The results of the products formed in Example 8 are shown in Table 2.

TABLE 2

| Tested Property | Units | Ex. 8 |
|---|---|---|
| Propylene produced | mg | 11 |
| 2-butene produced | mg | 128 |
| Ethane produced | mg | 13 |
| CHCl₃-soluble products | mg | 59 |
| Number average molecular weight (Mₙ) of product | g/mol | 2600 |
| distribution | | |
| Weight average molecular weight (M_{w}) of product | g/mol | 11000 |
| distribution | | |

### Example 9. Catalytic Conversion of Saturated Polyethylene in a Batch Reactor at Varied Reaction Times to Form Alkenes

In example 9, saturated polyethylene was reacted with ethylene over a first catalyst composition (CC 1) and a second catalyst composition (CC 2) in a 10 mL batch reactor (Parr reactor, Series 2500) according to Table 3. The reaction time was varied. In an N₂-filled glovebox, the first catalyst composition, the second catalyst composition, and the saturated polyethylene were loaded into a Parr 2500 reactor equipped with a pressure gauge and type J thermocouple. 28 psi of argon was added as an internal standard. Ethylene (99.999%, Airgas) was passed through a moisture/oxygen trap (Supelco) before use. Gas lines were purged of residual air for three 5-min cycles before ethylene was introduced into the reactor. Reactor heating was initiated, and reaction time was tracked after the desired temperature setpoint of 200° C. was reached. After the designated reaction time, the reactor was cooled in flowing air. Aliquots of gas from the reactor headspace were taken for GC analysis before venting the rest of the headspace in a fume hood. The remaining solid and liquid was transferred onto a fine glass filter (4.0-5.5 μm) and filtered to remove insoluble material by washing with hot (50° C.) CHCl₃. Soluble hydrocarbons were recovered by evaporating the solvent under reduced pressure (0.1 Torr). The insoluble material, including the catalyst and hydrocarbons insoluble in hot CHCl₃, was recovered from the filter. The results of the products formed in Examples 9A, 9B, and 9C are shown in Table 4.

TABLE 3

| | Saturated | Ethylene | | CC 1 | | CC 2 | Reaction | Reaction |
|---|---|---|---|---|---|---|---|---|
| | PE mass | Pressure | | mass | | mass | temperature | time |
| Example | (mg) | (psig) | CC 1 | (mg) | CC 2 | (mg) | (° C.) | (hours) |
| Ex. 9A | 120 | 339 | Ex. 5 | 200 | Ex. 2 | 201 | 200 | 3 |
| Ex. 9B | 120 | 340 | Ex. 5 | 206 | Ex. 2 | 200 | 200 | 6 |
| Ex. 9C | 121 | 340 | Ex. 5 | 201 | Ex. 2 | 201 | 200 | 9 |

TABLE 4

| Tested Property | Units | Ex. 9A | Ex. 9B | Ex. 9C |
|---|---|---|---|---|
| Propylene produced | mg | 31 | 11 | 8 |
| 2-butene produced | mg | 26 | 29 | 28 |
| Ethane produced | mg | 7 | 5 | 4 |
| CHCl₃-soluble products | mg | 38 | 25 | 8 |

### Example 10. Catalytic Conversion of n-Octadecane Over Various Catalyst Compositions in a Batch Reactor at Varied Reaction Times and Reaction Temperatures to Form Alkenes

In example 10, n-octadecane was reacted with ethylene over two catalyst compositions in a batch reactor (Parr reactor, Series 2500) according to Table 5. In an N₂-filled glovebox, a first catalyst composition (CC 1), a second catalyst composition (CC 2), and n-octadecane were loaded into a 10 mL reactor (Parr 2500) equipped with a pressure gauge and type J thermocouple. Ethylene (99.999%, Airgas) was passed through a moisture/oxygen trap (Supelco) before use. Gas lines were purged of residual air for three 5-min cycles before ethylene was introduced into the reactor. Reactor heating was initiated, and reaction time was tracked after the desired temperature setpoint was reached. After the designated reaction time, the reactor was cooled in flowing air. Aliquots of gas and liquids from the reactor headspace were taken for gas chromatography analysis with a flame ionization detector (GC-FID). The results of the products formed in Examples 1A-D and 1E-H are shown in Table 6 and Table 7, respectively.

TABLE 5

| | n-octadecane | Ethylene | | CC 1 | | CC 2 | Reaction | Reaction |
|---|---|---|---|---|---|---|---|---|
| | mass | Pressure | | mass | | mass | temperature | time |
| Example | (mg) | (psig) | CC 1 | (mg) | CC 2 | (mg) | (° C.) | (hours) |
| Ex. 10A | 398 | 260 | Ex. 1 | 129 | Ex. 2 | 200 | 100 | 6 |
| Ex. 10B | 103 | 242 | Ex. 1 | 102 | Ex. 2 | 101 | 150 | 6 |
| Ex. 10C | 104 | 237 | Ex. 1 | 104 | Ex. 2 | 100 | 150 | 18 |
| Ex. 10D | 107 | 237 | Ex. 1 | 101 | Ex. 2 | 99 | 150 | 24 |
| Ex. 10E | 212 | 708 | Ex. 1 | 99 | Ex. 3 | 101 | 130 | 24 |
| Ex. 10F | 198 | 709 | Ex. 1 | 100 | Ex. 4 | 101 | 130 | 24 |
| Ex. 10G | 212 | 707 | Ex. 1 | 98 | Ex. 2 | 101 | 130 | 24 |
| Ex. 10H | 74 | 238* | Ex. 1 | 39 | Ex. 6 | 34 | 130 | 6 |
| | | (mg) | | | | | | |

*Ethylene was added in Example 10H at a measured mass.

TABLE 6

| Tested Property | Units | Ex. 10A | Ex. 10B | Ex. 10C | Ex. 10D |
|---|---|---|---|---|---|
| Propylene produced | mg | 0.35 | 0.12 | 0.18 | 0.27 |
| 2-butene produced | mg | 0.24 | 0.39 | 0.62 | 1.1 |
| Ethane produced | mg | 0 | 0.04 | 3.0 | 4.0 |

TABLE 7

| Tested Property | Units | Ex. 10E | Ex. 10F | Ex. 10G | Ex. 10H |
|---|---|---|---|---|---|
| Propylene produced | mg | 17 | 4 | 36 | 3 |
| 2-butene produced | mg | 13 | 12 | 16 | 1.5 |

### Example 11. Catalytic Conversion of Saturated Polyethylene in a Flow Reactor to Form Alkenes

In Example 11, a flow reactor was used, as shown in FIG. 1. A 20 mL glass reaction sleeve (ID=19.56 mm, OD=22.15 mm) was placed into a 40 mL stainless-steel stirred-tank reactor (ID=22.16 mm, OD=40 mm), and the reactor was housed within an aluminum heating jacket. The temperature of the heating jacket was controlled by a hotplate and thermocouple (IKA C-MAG HS7 digital). The reactor has two inlet ports, one for liquid substrates and a second for gaseous substrates. Liquid substrates were delivered into the setup using a Hamilton gas-tight syringe (5 mL) and a Kd Scientific Legato 100 Syringe Pump. Gaseous substrates were supplied from a pressurized tank whose flow was set by an Alicat mass flow controller (MCS series). The outlet stream led to an Equilibar backpressure regulator which was used to control the reaction pressure. Attached downstream from the regulator was an Agilent 6850 gas chromatograph (GC). The GC was equipped with a 6-port VICI-Valco gas-sampling valve. A continuous flow of ethylene was used as an internal standard to quantify olefin formation rates. Stainless-steel tubing and fittings were purchased from McMaster-Carr and Swagelok. The GC was equipped with an FID and a Petrocol DH Capillary GC Column (100 mm×0.25 mm×0.5 μm film thickness). The column was held at 45 PSI and the gaseous sample was split 50:1. The column conditions and product elution times are shown in Table 8 and Table 9, respectively.

TABLE 8

| Oven Ramp | ° C./min | Next ° C. | Hold min |
|---|---|---|---|
| Initial | | 50 | 8 |
| Ramp1 | 15 | 100 | 1 |
| Ramp2 | 15 | 150 | 15.00 |

TABLE 9

| Hydrocarbon Species | Elution Time min |
|---|---|
| Ethylene | 8.2 |
| Propylene | 8.3 |
| Butenes | 8.8-9.0 |
| Pentenes | 10.1-9.5 |
| Hexenes | 12.2-14.1 |
| Heptenes | 15.1-17.0 |
| Octenes | 19.0-20.1 |
| Nonenes | 22.0-24.1 |
| Decenes | 25.4-26.3 |

Within an Ar-filled glovebox, 249 mg of saturated polyethylene, 146 mg of Example 1, and 36.6 mg of Example 7 were loaded into a stirred-tank reactor. After loading, the reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. For 19.5 hours, ethylene gas (10.1 mL/min) was continuously flown into the reactor which was heated to and held at 130° C. and 1 atm. To monitor reaction progression, a sample of the gaseous effluent (0.25 mL) was analyzed via GC every 34.2 minutes for the 19.5-hour duration of the reaction. The results of Example 11 are shown in Table 10. The maximum propylene formation rate detected while catalyst is on-stream (R_{C3, max}), in millimoles per hour (mmol h⁻¹), was measured. The maximum propylene selectivity (S_{C3}, max) while the catalyst was on-stream was measured. The formation rate of propylene is normalized by the cumulative olefin formation rate for a given reaction time. The average of the selectivity of propylene (S_{C3,avg}) and butylene (S_{C4,avg}) were evaluated at each sampling point during the course of the continuous reaction for each species. The polyethylene conversion, in weight percent, was also estimated by calculating the mass of polyethylene consumed per olefin produced, according to equation 1:

$$\frac{Σ ( \frac{i - 2}{i} {n}_{Ci} {MW}_{i} ) * 100}{{m}_{PE , o}} \qquad ( 1 )$$

where i represents the number of carbon units within the olefin, MWᵢ represents the molecular weight of species “i”, n_{ci} is the moles of species “i” formed during the experiment, and m_{PE,o} is the initial loading of polyethylene, assuming each molecule of olefin formed contains two carbons from ethylene.

TABLE 10

| Olefins | R_{C3, max}/ | | | | PE |
|---|---|---|---|---|---|
| Detected | mmol h⁻¹ | S_{C3, max} | S_{C3, avg} | S_{C4, avg} | Conversion |
| C₃-C₄ | 0.04 | 0.83 | 0.72 | 0.28 | 1.2 wt. % |

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter of the present disclosure has been described in detail and by reference to specific embodiments. It should be understood that any detailed description of a component or feature of one or more embodiments does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A process for converting saturated polyethylene to at least an alkene product of chemical formula CₘH₂ₘ, the process comprising contacting the saturated polyethylene with three or more catalyst components in a reactor, the reactor comprising an alkene reactant of chemical formula CₙH₂ₙ;
where:
m is an integer from 3 to 20
n is an integer from 2 to 20;
the three or more catalyst components comprise a metathesis catalyst component, an isomerization catalyst component, and a dehydrogenation catalyst component; and
contacting causes at least a portion of the saturated polyethylene to undergo dehydrogenation reactions to form unsaturated polyethylene and at least a portion of the unsaturated polyethylene, or products derived therefrom, to undergo metathesis reactions and isomerization reactions to produce an effluent comprising at least the alkene product of chemical formula CₘH₂ₘ.

2. The process of claim 1, wherein a pressure of the alkene reactant in the reactor during the contacting is from 0 pounds per square inch gauge (psig) to 3000 psig.

3. The process of claim 1, wherein a temperature of the reactor during the contacting is less than or equal to 400° C.

4. The process of claim 1, wherein the alkene reactant comprises ethylene, propylene, butenes, pentenes, or combinations thereof.

5. The process of claim 1, wherein the alkene product comprises propylene, butenes, pentenes, or combinations thereof.

6. The process of claim 1, wherein the metathesis catalyst component comprises an element selected from International Union of Pure and Applied Chemistry (IUPAC) groups 5-10.

7. The process of claim 1, wherein the metathesis catalyst component comprises rhenium, ruthenium, tungsten, molybdenum, vanadium, or combinations thereof.

8. The process of claim 1, wherein the metathesis catalyst component comprises methyltrioxorhenium (MTO).

9. The process of claim 1, wherein the isomerization catalyst component comprises an element selected from IUPAC groups 5-10.

10. The process of claim 1, wherein the isomerization catalyst component comprises alumina, silica, iridium, palladium, ruthenium or combinations thereof.

11. The process of claim 1, wherein the dehydrogenation catalyst component comprises an element selected from IUPAC groups 5-10.

12. The process of claim 1, wherein the dehydrogenation catalyst component comprises platinum, iridium, ruthenium, rhenium, or combinations thereof.

13. The process of claim 1, wherein a first catalyst composition comprises the metathesis catalyst component and the isomerization catalyst component, and wherein the first catalyst composition comprises MTO on alumina.

14. The process of claim 1, wherein a second catalyst composition comprises the isomerization catalyst component and the dehydrogenation catalyst component, and wherein the second catalyst composition comprises platinum on alumina, platinum on silica, [tert-butyl-POCOP]Ir[C₂H₄] or combinations thereof.

15. The process of claim 1, wherein a first catalyst composition comprising MTO on alumina and a second catalyst composition comprising platinum on alumina contact the saturated polyethylene in the reactor.
